(19) United States
(12) Patent Application Publication
(10) Pub. No.: US 2009/0035310 A1
(43) Pub. Date: Feb. 5, 2009

(54) CANCER TREATMENT

(75) Inventors: Arun Rishi, Detroit, MI (US); Liyue Zhang, Detroit, MI (US)

Correspondence Address: DAVIS WRIGHT TREMAINE, LLP/Seattle, 1201 Third Avenue, Suite 2200, SEATTLE, WA, 98101-3045, US

(73) Assignee: WAYNE STATE UNIVERSITY, Detroit, MI (US)

(21) Appl. No.: 12/280,980

(22) PCT Filed: Feb. 27, 2007
(86) PCT No.: PCT/US07/62914

§ 371 Date: Aug. 27, 2008

Related U.S. Application Data
(60) Provisional application No. 60/777,427, filed on Feb. 28, 2006.

(51) Int. Cl. A61K 48/00; A61K 38/17; A61K 39/395; C12N 5/06; A61K 31/7088; A61P 35/00; C12N 15/85; C12N 15/861

(52) U.S. Cl. 424/133.1; 514/12; 435/375; 435/455; 514/44; 424/93.2

(57) ABSTRACT

Pharmaceutical compositions and methods for inhibiting tumor growth and/or for inducing apoptosis are provided. Such compositions comprise at least one polypeptide of contiguous amino acids 1-198, 197-454, or 896-1150 of CARP-1 protein, and derivatives thereof, and may optionally comprise one or more cancer chemotherapeutic agents. Also provided are plasmids encoding the polypeptides, and methods of using the polypeptides and plasmids to inhibit cancer cell growth, including in mammals.

### CROSS-REFERENCE TO RELATED APPLICATION

This application claims priority to provisional application Ser. No. 60/777,427, filed Feb. 28, 2006, which is incorporated by reference herein.

### STATEMENT REGARDING FEDERALLY SPONSORED RESEARCH OR DEVELOPMENT

This work was supported by a VA Merit Grant from Department of Veterans Affairs, a Karmanos Cancer Institutional Research Grant from Children's Leukemia foundation of Michigan, and a Detroit Medical Center Institute for Oncology and Allied Diseases (DMCIOAD) Institutional Research Grant.

### FIELD OF THE INVENTION

The invention disclosed herein relates to compositions and methods useful for the diagnosis and treatment of cancer.

### BACKGROUND OF THE INVENTION

Programmed cell death, or apoptosis, is essential for the development and maintenance of cellular homeostasis. The pathways regulating apoptosis serve as important targets for many anticancer agents currently utilized for treatment of diverse malignancies, including breast cancer. Despite years of research, there remains a pronounced need in the art for new methods of diagnosing and treating cancer, including targeting biochemical pathways relating to apoptosis.

### BRIEF SUMMARY OF THE INVENTION

The invention disclosed herein relates to compositions of CARP-1 proteins and polypeptides and methods useful for the diagnosis and treatment of cancer.

Also provided is a pharmaceutical composition comprising a polypeptide consisting of a polypeptide fragment of CARP-1, in admixture with a pharmaceutically acceptable carrier. The polypeptide fragment may consist of contiguous amino acids 1-198, 197-454, or 896-1150 of CARP-1 (SEQ ID NO:5).

The pharmaceutical composition may further comprise at least one cancer chemotherapeutic agent, such as adriamycin or Herceptin.

Also provided is a method of inhibiting cancer cell growth in a mammalian subject in need thereof, comprising administering to the subject a therapeutically effective amount of a pharmaceutical composition comprising a polypeptide consisting of a polypeptide fragment of CARP-1, in admixture with a pharmaceutically acceptable carrier composition. The polypeptide fragment may consist of contiguous amino acids 1-198, 197-454, or 896-1150 of CARP-1. The mammalian subject may be a human afflicted with breast cancer, a human afflicted with colon cancer, or a domestic or agricultural animal.

Further provided is a method of inducing apoptosis in a cancer cell, comprising administering to the cancer cell a polypeptide fragment of CARP-1, such as a polypeptide consisting of contiguous amino acids 1-198, 197-454, or 896-1150 of CARP-1.

Another method of inducing apoptosis in a cancer cell comprises administering to the cancer cell a vector comprising a polynucleotide encoding a polypeptide consisting of contiguous amino acids 1-198, 197-454, or 896-1150 of CARP-1, wherein the vector is capable of directing expression of the polypeptide in the cell. The vector may be an AAV vector.

Further provided is a method of inhibiting cancer cell growth in a mammalian subject in need thereof, comprising administering a therapeutically effective amount of a vector comprising a polynucleotide encoding a polypeptide consisting of contiguous amino acids 1-198, 197-454, or 896-1150 of CARP-1, wherein the vector is capable of directing expression of the polypeptide in the subject. The vector may be an AAV vector.

For the methods herein, the mammalian subject may be a human afflicted with cancer of a type including but not limited to breast cancer, colon cancer, lung cancer, prostate cancer, liver cancer, kidney cancer, or bladder cancer.

### BRIEF DESCRIPTION OF THE DRAWINGS

FIG. 1 shows that CARP-1 expression alters biological properties of HBC cells. (A) Western blot showing expression of CARP-1-myc-His fusion protein in MDA-MB-468 (wt), and clone 6.1 sublines 1-3 HBC cells. Protein lysate (100 μg) from each subline was analyzed on 10% SDS-PAGE using anti-His or anti-actin antibodies. (B) Histogram showing relative number of colonies of indicated cell types growing in soft agar. Columns represent means of three independent experiments, bars, SE. (C) Each of the indicated cell type were separately seeded in chambers having matrigel-coated membranes, followed by staining of the cells migrating through membranes. Histogram represents the average cell counts from multiple, independent fields having stained HBC cells in two independent experiments. (D) MDA-MB-468 (wt), vector transfected subline, and clone 6.1 sublines 1, 3 HBC cells were grown as tumors in SCID mice. Histogram shows HBC cell-derived tumor weight (in mg; mean+/−SD) in SCID mice. The data in panel D was derived from two independent experiments.

FIG. 2A-C. FIG. 2A shows diagrammatic maps of various CARP-1 peptides and eGFP having indicated epitopes positioned at their amino termini. CARP-1 amino acid numbers are indicated above the respective bar for each construct. Construct names are indicated on the right. FIG. 2B shows the nucleotide (SEQ ID NO:1) and amino acid sequence (SEQ ID NO:2) for His-TAT-HA-CARP-1 1-198. For the polynucleotide, CARP-1 cDNA is underlined, restriction sites are bold and underlined. The translation start and termination codons are in bold and italics. For the amino acid sequence, bold italics indicate the His tag; italics underlined indicate the TAT epitope (YGRKKRRQRRR); and bold italics underlined indicate the HA tag (YPYDVPDYA). CARP-1 1-198 is underlined. FIG. 2C shows the nucleotide (SEQ ID NO:3) and amino acid sequence (SEQ ID NO:4) for His-TAT-HA-CARP-1 896-1150. For the polynucleotide, CARP-1 cDNA is underlined, and restriction sites are bold and underlined. The translation start and termination codons are in bold and italics. For the amino acid sequence, bold italics indicate the His tag; italics underlined indicate the TAT epitope (YGRKKRRQRRR); and bold italics underlined indicate the HA tag (YPYDVPDYA). CARP-1 896-1150 is underlined.

FIG. 3 shows the purification of TAT-tagged peptides. E. coli was transformed with various recombinant plasmids. Logarithmically growing cells were induced with 100 μM IPTG for 4 h, cell pellets harvested, and lysed. The lysates were incubated with ProBond resin, and recombinant proteins eluted with Imidazole following manufacturer's guidelines. Aliquots from washes and eluted fractions were electrophoresed on a 12% SDS-PAGE gel, followed by staining of gel with Coomassie blue. The arrows in each lane indicate the purified protein/peptide, while the molecular mass (kDa) of the select protein ladder are noted on the left.

FIG. 4A-F shows HBC cell transduction by TAT-tagged proteins. HBC cells were incubated with 50 μg/ml of affinity-purified respective proteins for a period of 24 hours. Immunocytochemical staining of the transduced cells was carried out by utilizing anti-HA-tag antibodies. Presence of the transduced peptides is indicated by darker staining in the cytoplasmic region in panels A-E. In panel F, only viable live cells were spun down and stained with HA-tag antibody without prior incubation with anti-mouse secondary antibodies.

FIG. 5 shows cell growth inhibition and apoptosis induction by CARP-1 peptides. HBC cells were incubated with the noted doses of each peptide for 48 hours, and the cell lysates were prepared for MTT assay (A) or for determination of apoptosis (B). The columns represent means of three independent experiments; bars, SE.

FIG. 6 shows apoptosis signaling by CARP-1 peptides involves activation of p38 α/β SAPK (A) and caspase-9 (B). Western immunoblots of HBC cells that were either untreated (Control) or treated with the indicated peptides as in FIG. 5 for 48 hours. Protein lysate (100 μg) from each sample was analyzed on 10% SDS-PAGE followed by immunoblotting with anti-phospho-p38, p38, or caspase-9 antibodies.

FIG. 7 shows the effect of His-TAT-HA-CARP-(1-198) on growth of WSU-DLCL₂ chemotherapy resistant lymphoma cells. Cells were either untreated (Control), or treated, in vitro, with 150 μg/mL affinity purified peptides for noted times. The cells were stained with trypan blue, and number of live, viable cells determined at the end of each treatment period. The columns represent mean determination of four experiments; bars, SE.

FIGS. 8A and B. FIG. 8A shows HBC cells that were grown as tumors in SCID mice, and the tumors were treated with affinity purified peptides as detailed in the text. The histogram shows HBC cell-derived tumor weight (in mg; mean+/−SD) in SCID mice that were treated with noted peptides. FIG. 8B shows that His-TAT-HA-CARP-1 (1-198) as well as (896-1150) proteins inhibited growth of drug-resistant WSU-DLCL₂ lymphoma cell-derived tumor xenografts in SCID mice.

FIG. 9 shows immunostaining of tumors for presence of CARP-1 and eGFP proteins (column with anti-HA-tag antibody) or activated p38 protein (column with anti-phospho-p38 antibody) in untreated (Control) or treated SCID mice. Presence of the transduced peptides and activation of p38 is reflected by darker staining in the respective panels.

FIG. 10A-F shows immunostaining of tumors for presence of CARP-1 as detected using anti-CARP-1 antibody. FIGS. 10A and B, liver; 10C and D, kidney; and 10E and F, bladder. Representative higher power (400×) micrographs of poorly differentiated tumors (panels B, D, F) and the adjoining normal tissues (panels A, C, E) are presented.

FIG. 11A-H shows immunostaining of tumors for presence of CARP-1 as detected using anti-CARP-1 antibody. FIGS. 11A and B, breast; 11C and D, colon; 11E and F, lung; and 11G and H, prostate. Representative higher power (400×) micrographs of poorly differentiated tumors (panels B, D, F, H) and the adjoining normal tissues (panels A, C, E, G) are presented.

### DETAILED DESCRIPTION OF THE INVENTION

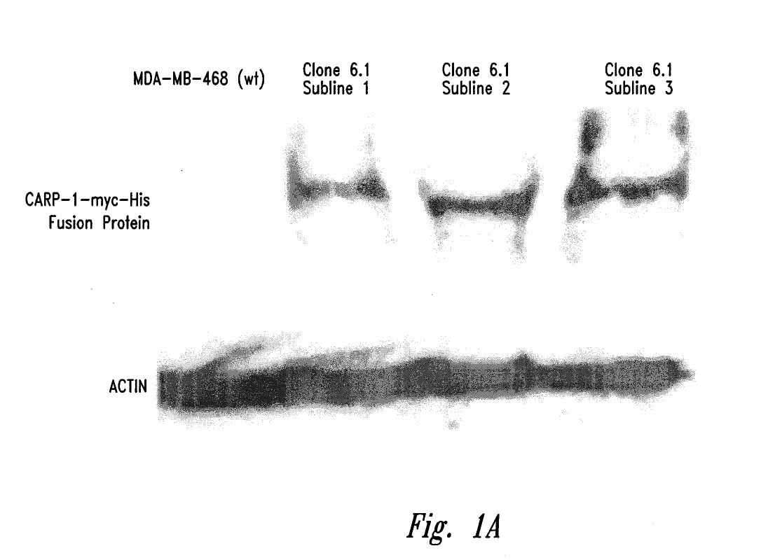

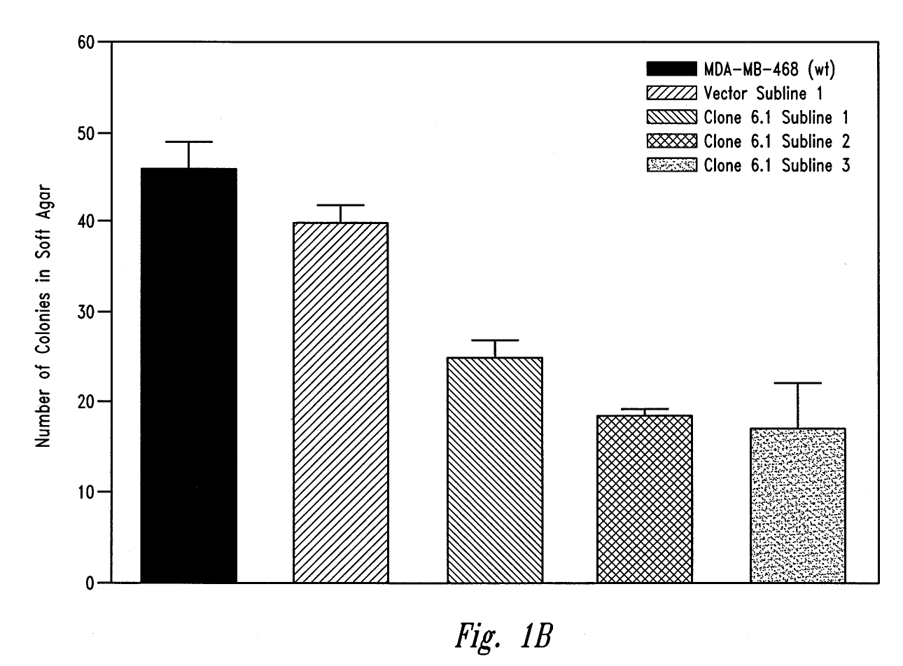

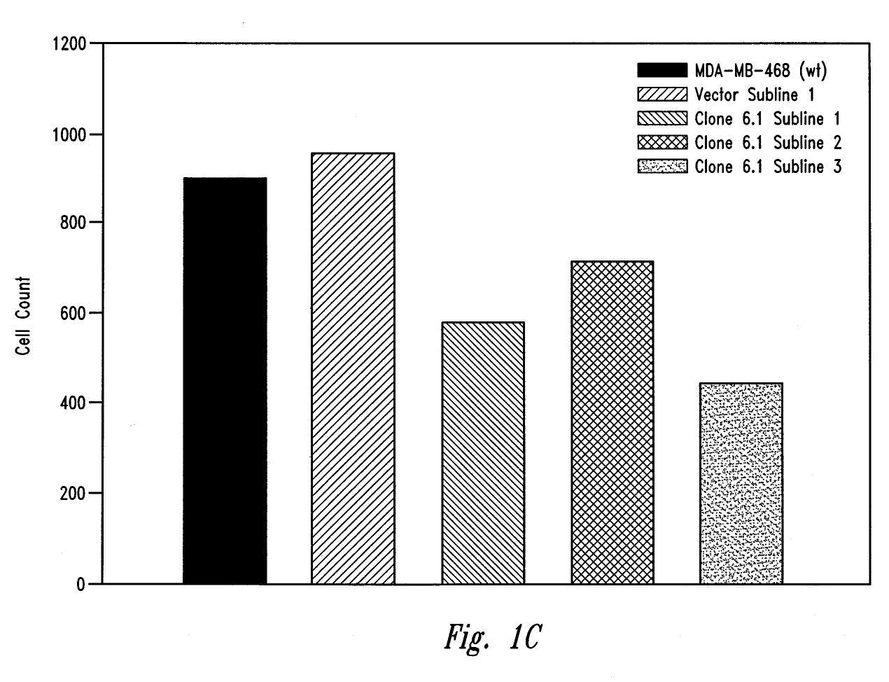

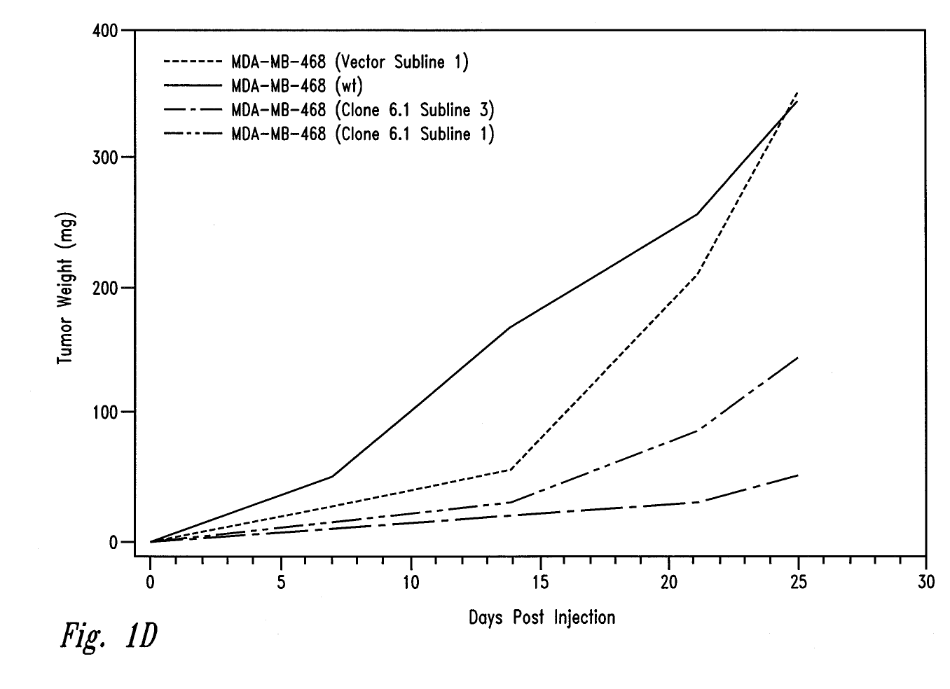

CARP-1 is a recently-identified 130 kDa protein (Rishi, A. K et al., J. Biol. Chem. 278: 33422-33435, 2003). The full length amino acid sequence of CARP-1 is shown in SEQ ID NO:5, and the corresponding polynucleotide sequence is shown in SEQ ID NO:6. According to the invention, CARP-1 proteins and polypeptides caused apoptosis and cell growth inhibition of human breast and colon cancer cells. Over-expression of CARP-1 in human breast cancer (HBC) cells inhibited formation of tumors in SCID mice, indicating a tumor-suppressor property of CARP-1 (FIG. 1). CARP-1 expression correlated inversely with the grades of human breast and colon cancers (Tables 1 and 2). In diffuse large B-Cell lymphomas, CARP-1 expression correlated with cleaved caspase-3 while an inverse correlation was noted between its expression and the presence of phospho-Akt, BCI2, and MUM1 (Table 3).

The observations in Tables 1-3 also indicate prognostic and diagnostic uses for this protein. Table 1 shows CARP-1 expression in human breast cancers (N=100). In the results shown in Table 1, CARP-1 expression inversely correlated with the Tumor Histologic Grade (Fisher's exact P value=0.00000378).

TABLE 1

CARP-1 Expression

| Tumor Histologic Grade | Low | High |
|---|---|---|
| Low | 16 | 27 |
| High | 47 | 10 |

Table 2 shows CARP-1 expression in human colon cancers (N=56). In the results shown in Table 2, CARP-1 expression inversely correlated with the Tumor Histologic Grade (P=0.0015).

TABLE 2

CARP-1 Expression

| Tumor Histologic Grade | Low | High |
|---|---|---|
| I | 8 | 11 |
| II + III | 29 | 8 |

Table 3 shows a correlation of CARP-1 expression with cleaved caspase-3, p-Akt, Bcl6, Bcl2, CD10 and IRF-4/MUM1 expression in diffuse large B-cell lymphomas (n=83).

TABLE 3

| | | Cleaved | | | | | IRF-4/ |
|---|---|---|---|---|---|---|---|
| | | caspase-3 | p-Akt | Bcl6 | Bcl2 | CD10 | MUM1 |
| CARP- | CC | 0.355 | −0.240 | 0.274 | −0.111 | 0.090 | −0.172 |
| 1 | P | 0.001* | 0.029* | 0.012* | 0.323 | 0.417 | 0.119 |
| | value | | | | | | |

CARP-1 has been shown to be a regulator of apoptosis signaling, and CARP-1 expression plays an important role in apoptosis induction in human breast cancer (HBC) cells in the presence of chemotherapy agent adriamycin. According to the invention, CARP-1 is a regulator of apoptosis that is also induced when proliferation signaling by epidermal growth factor receptor (EGFR) is inhibited. EGFR protein family members are known risk factors for development of neoplastic disease, and are predictors of prognosis and response to therapy. In addition, they may be utilized as therapeutic targets against a variety of cancers including breast cancer, as it is likely that expression of CARP-1 will sensitize cancer cells to growth inhibition by anti-cancer agents that specifically target EGFR function.

Further, it has been demonstrated previously that chemotherapy agents such as adriamycin-dependent growth inhibition and apoptosis of HBC cells involve elevated expression of CARP-1. Because apoptosis-inducing CARP-1 peptide (His-TAT-HA-CARP-1 1-198; SEQ ID NO:2) inhibited growth of HBC cells in vitro as well as HBC cell-derived tumor growth in vivo, treatment of breast cancers with a combination of His-TAT-HA-CARP-1 1-198 protein and agents like adriamycin or Herceptin may be superior in inhibition/killing of breast cancer.

Since small molecules (synthetic and/or peptides) are preferred as anti-cancer therapeutic agents for efficacious delivery, a CARP-1 peptide has been identified that is significantly smaller in size (˜27-30 kDa) and functions as inducer of apoptosis and inhibitor of HBC cell growth both in vitro and in vivo. Discovery of apoptosis-inducing His-TAT-HA-CARP-1 1-198 protein may facilitate CARP-1 utilization as an efficacious anti-tumor agent for treatment of breast cancer alone or in conjunction with chemotherapy agents such as adriamycin, Herceptin.

Aspects of the invention relate to the development of CARP-1 or its apoptosis-inducing peptide(s) for utilization as therapeutic agents against cancer, including human breast cancer. In light of the fact that wild-type CARP-1 protein is 130 kDa in size, apoptosis-inducing domain(s) of CARP-1 would allow generation of smaller sized CARP-1 mutants with pro-apoptotic and cancer cell growth inhibitory properties. Such domains are within the scope of this disclosure, and induction of apoptosis can be routinely assayed as described in Example 3, for example.

To this end, a recombinant plasmid that encodes a mutant CARP-1 protein was generated. This plasmid-derived mutant CARP-1 protein has amino acids 1-198 of the wild-type CARP-1 protein (amino acid 1 being starting Methionine in wild-type CARP-1 protein). In addition, this CARP-1 1-198 mutant protein possesses epitopes for TAT-based transduction and His, HA-tag positioned at the amino-terminus. This recombinant, His-TAT-HA-CARP-1 1-198 fusion protein was affinity purified from E. coli extracts and utilized to transduce HBC cells.

Transduction of HBC cells with His-TAT-HA-CARP-1 1-198 protein results in inhibition of their growth as well as elevated levels of apoptosis (see Examples). This protein, along with its derivative that carries a substitution of tyrosine 192 to phenylalanine (His-TAT-HA-CARP-1 1-198Y192>F), and His-TAT-HA-enhanced green fluorescent proteins was utilized to treat HBC cell-derived tumor xenografts in SCID mice. As also shown below in the Examples, the data revealed that intratumoral injections of His-TAT-HA-CARP-1 1-198, but not His-TAT-HA-CARP-1 1-198Y192>F or His-TAT-HA-eGFP, caused significant inhibition of the growth of the tumor xenografts.

The Examples below describe methods of making and using this and other CARP-1 polypeptides. Other polypeptides include CARP-1 197-454, and CARP-1 896-1150, with the number ranges representing amino acid positions in SEQ ID NO:5. CARP-1 is a regulator of apoptosis signaling by epidermal growth factor receptor family of proteins. As CARP-1 is a potential tumor-suppressor, CARP-1 protein or its derivatives may be of value as anti-cancer agents, including anti-breast cancer agents. To test this possibility, multiple, non-overlapping CARP-1 peptides were generated and utilized to study inhibition of human breast cancer (HBC) cell growth in vitro and in vivo. Each peptide harbors amino-terminal epitopes for polyhistidine, HA tags for detection and purification, and retroviral TAT-domain for transport of the fusion proteins into cells.

Incubation of MDA-MB-468 HBC cells with His-TAT-HA-CARP-1 peptides revealed predominantly perinuclear localization of all the peptides. Treatment of HBC as well as COS-7 monkey kidney cells, in vitro, with His-TAT-HA-CARP-1 1-198, 197-454, or 896-1150 caused inhibition of cell proliferation and elevated apoptosis. The cells that were treated with affinity purified His-TAT-HA-tagged enhanced green fluorescent (eGFP) protein or His-TAT-HA-CARP-1 (1-198) peptides that have a substitution at tyrosine¹⁹² to phenylalanine, on the other hand, failed to show inhibition of cell proliferation or induction of apoptosis. Apoptosis induction by CARP-1 peptides, with the exception of CARP-1 (1-198; Tyr¹⁹²/Phe) involves increased phosphorylation of stress-activated protein kinase (SAPK) p38, and activation of caspase-9.

Expression of wild-type CARP-1 interferes with the ability of HBC cells to invade through the matrigel coated membranes, to form colonies in the soft agar, and to grow as tumors in the SCID mice. Moreover, intratumoral injections of His-TAT-HA-CARP-1 (1-198), but not His-TAT-HA-eGFP or His-TAT-HA-CARP-1 (1-198; Y192>F), inhibits growth of HBC cell-derived tumor xenografts in SCID mice. Together, the data suggest that apoptosis induction by CARP-1 contributes, in part, to the tumor suppressor properties of CARP-1 in HBC cells.

CARP-1 is an apoptosis-inducing protein identified from human breast cancer cells. As noted above, CARP-1 expression correlates inversely with grades of breast and colon cancers. To test the toxicity of CARP-1 in mice, affinity purified His-TAT-HA-CARP-1 1-198 peptide was injected by tail vein at a dose of 5 mg/kg/day for four consecutive injections in SCID mice. In addition, SCID mice were injected subcutaneously (s.c.) His-TAT-HA-CARP-1 1-198 peptide or His-TAT-eGFP peptide at a dose of 17 mg/kg/day or 14 mg/kg/day, respectively, for 10 injections. Both the treatments did not cause any visible toxicity including symptoms of diarrhea, dehydration, weight loss, hair loss, or any other discomfort. This observation suggests that His-TAT-HA-CARP-1 1-198 peptide may be non-toxic and fairly well tolerated.

The TAT tag belongs to a class of peptide molecular transporter domains (also known as protein transduction domains, PTDs). PTDs are 10-30 amino acid residues in length and are rich in basic amino acids, e.g., arginine and lysine. Transduction of proteins bearing PTDs does not depend on classical receptor-mediated, transmembrane, or endocytic pathways and is a novel and emerging strategy to transport proteins across the plasma membrane of eukaryotic cells. Many TAT-fusion proteins of varying sizes and functions have been successfully transported inside the normal and tumor cells in vitro, as well as delivered to the desired sites in experimental mouse models in vivo.

CARP-1 polypeptides. Although the specification herein discloses particular CARP-1 polypeptides (amino acids 1-198; 197-454; and 896-1150 of SEQ ID NO:5), due to the degeneracy of nucleotide coding sequences, other polynucleotide sequences which encode substantially the same amino acid sequence as a CARP-1 polynucleotide (such as SEQ ID NO:6) may be used to generate suitable polypeptides, including those comprising conservative substitutions. The resulting polypeptides are tested for biological function as described below. These polynucleotide sequences include but are not limited to modified allelic genes, modified homologous genes from other species, and nucleotide sequences comprising all or portions of CARP-1 genes which are altered by the substitution of different codons that encode the same amino acid residue within the sequence, thus producing a silent change. Likewise, the CARP-1 polypeptides of the invention can include, but are not limited to, those containing, as a primary amino acid sequence, all or part of the amino acid sequence of a CARP-1 polypeptide (such as amino acids 1-198, amino acids 197-454, or amino acids 896-1150 of SEQ ID NO:5) including altered sequences in which functionally equivalent amino acid residues are substituted for residues within the sequence resulting in a conservative amino acid substitution. Such substitutions are defined as a conservative substitution.

For example, one or more amino acid residues within the CARP-1 polypeptide sequence can be substituted by another amino acid of a similar polarity, which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. Amino acids containing aromatic ring structures are phenylalanine, tryptophan, and tyrosine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Such alterations are not expected to significantly affect apparent molecular weight as determined by polyacrylamide gel electrophoresis, or isoelectric point.

Suitable conservative substitutions are: Lys for Arg and vice versa such that a positive charge may be maintained; Glu for Asp and vice versa such that a negative charge may be maintained; Ser for Thr such that a free —OH can be maintained; and Gln for Asn such that a free NH₂ can be maintained.

Amino acid substitutions may also be introduced to substitute an amino acid with a particularly preferable property. For example, a Cys may be introduced at a potential site for disulfide bridges with another Cys. Pro may be introduced because of its particularly planar structure, which induces β-turns in the protein's structure.

Polypeptides having such conservative amino acids substitutions and relevant biological function are within the scope of this disclosure and claims. Relevant biological function of these CARP-1 polypeptides (including induction of apoptosis, and inhibition of cancer cell growth in vivo and in vitro) can be assayed as taught herein. For example, induction of apoptosis can be routinely assayed as described in Example 3. Inhibition of cancer cell growth can be routinely assayed as described in Examples 3-5.

Pharmaceutical compositions. The present invention is directed in part towards methods for inhibiting tumor growth and/or inducing apoptosis in tumor cells by the administration of a polypeptide fragment of CARP-1. The polypeptide fragment may consist of contiguous amino acids 1-198, 197-454, or 896-1150 of CARP-1, and the polypeptide fragment may optionally be administered with at least one anticancer agent such as adriamycin or Herceptin. These agents can be administered separately (e.g, formulated and administered separately), or in combination as one pharmaceutical composition as is known in the art, for example, as disclosed in references cited in U.S. Pat. No. 7,037,906. Administration can be achieved by any suitable route, such as parenterally, transmucosally, e.g., orally, nasally, or rectally, or transdermally. Alternative means of administration also include, but are not limited to, intra-arteriole, intramuscular, intradermal, subcutaneous, intraperitoneal, intraventricular, and intracranial administration, or by injection into the tumor(s) being treated or into tissues surrounding the tumor(s). The CARP-1 polypeptide fragment of the invention can be administered as a polypeptide or via a vector, wherein in certain embodiments, the vector is comprised of a polynucleotide encoding contiguous amino acids 1-198, 197-454, or 896-1150 of CARP-1. One suitable vector is AAV.

A CARP-1 polypeptide of the invention and the one or more optional anticancer agent may be employed in any suitable pharmaceutical formulation, as described above, including in a vesicle, such as a liposome as described, for example, in Langer, Science 249:1527 1533 (1990); Treat et al., in Liposomes in the Therapy of Infectious Disease and Cancer, Lopez-Berestein and Fidler (eds.), Liss: New York, pp. 317-327. Preferably, administration of liposomes containing the agents of the invention is parenteral, e.g., via intravenous injection, but also may include, without limitation, intra-arteriole, intramuscular, intradermal, subcutaneous, intraperitoneal, intraventricular, and intracranial administration, or by injection into the tumor(s) being treated or into tissues surrounding the tumor(s). A suitable and non-limiting subcutaneous dose range includes 15-17 mg/kg for ten days. A suitable and non-limiting intravenous dose range includes 5 mg/kg for three days.

In another embodiment, a pharmaceutical composition of the present invention can be delivered in a controlled release system, such as using an intravenous infusion, an implantable osmotic pump, a transdermal patch, liposomes, or other modes of administration. In a particular embodiment, a pump may be used (Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (Medical Applications of Controlled Release, Langer and Wise (eds.), CRC Press: Boca Raton, Fla. (1974); Controlled Drug Bioavailability, Drug Product Design and Performance, Smolen and Ball (eds.), Wiley: New York (1984); Ranger and Peppas, J. Macromol. Sci. Rev. Macromol. Chem. 23:61 (1983); Levy et al., Science 228:190 (1985); During et al., Ann. Neurol. 25:351 (1989); Howard et al., J. Neurosurg. 71:105 (1989)).

In a further embodiment, a controlled release system can be placed in proximity of the target tissues of the animal, thus requiring only a fraction of the systemic dose (Goodson, in Medical Applications of Controlled Release, supra, vol. 2, pp. 115 138 (1984)). In particular, a controlled release device can be introduced into an animal in proximity of the site of a tumor. Other controlled release systems are discussed in Langer (Science 249:1527 1533 (1990)).

The treatment methods and compositions herein are suitable for animals, particularly mammals in need thereof, including humans, and also including domestic animals such as dogs and cats, and agricultural animals such as pigs, sheep, cattle, and horses.

The following Examples are included for purposes of illustration only, and are not intended to limit the scope of the range of techniques and protocols in which the compositions and methods of the present invention may find utility, as will be appreciated by one of skill in the art and can be readily implemented.

### EXAMPLES

### Example 1

### Purification of Recombinant HIS-Tat-HA-CARP-1 Proteins

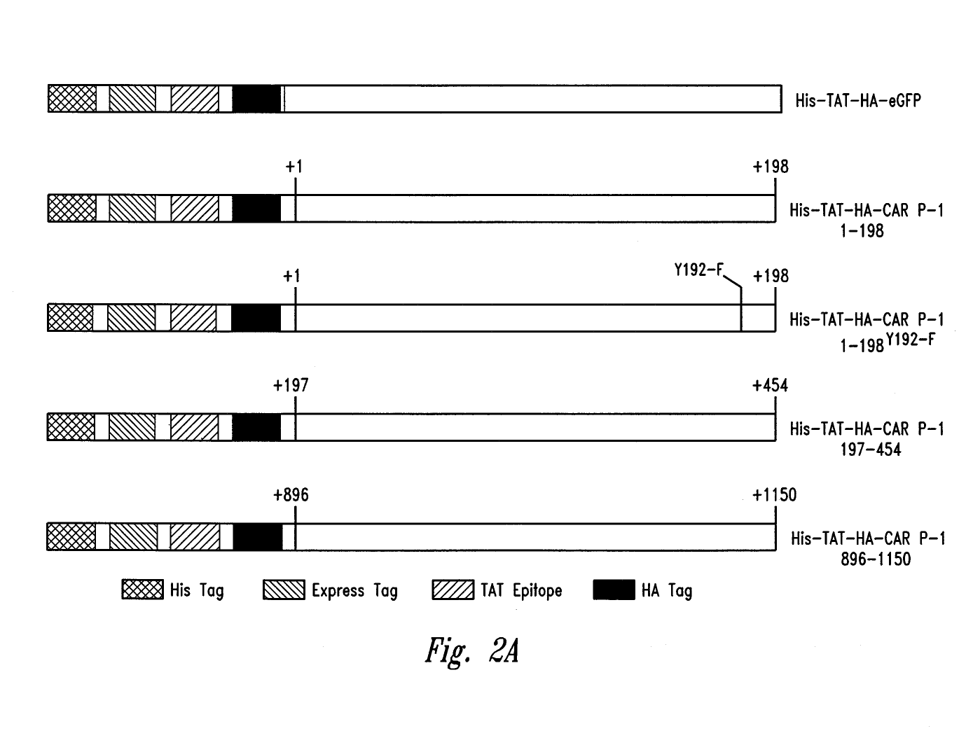

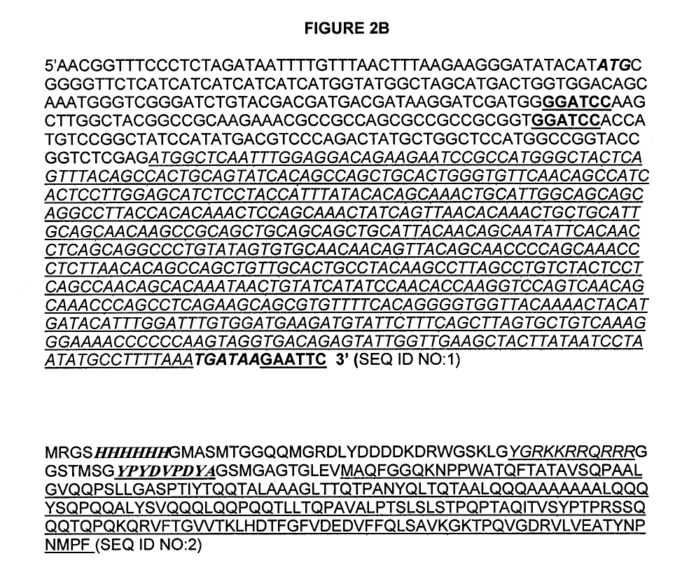

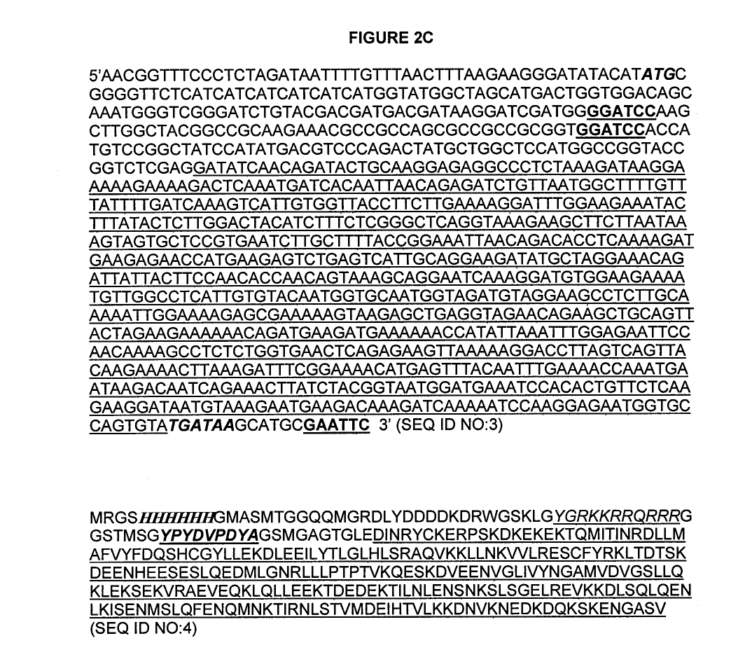

CARP-1 cDNA fragments for expression of CARP-1 1-198, 197-454, and 896-1150 peptides were PCR amplified using wild-type CARP-1 cDNA (GenBank ref. # AY249140) as template. These fragments were separately subcloned in vector plasmid pTAT/HA to obtain constructs for expression of various His-TAT-HA-tagged peptides (shown schematically in FIG. 2). In addition, a plasmid encoding His-TAT-HA-CARP-1 1-198 derivative having substitution of tyrosine 192 to phenylalanine was generated by standard molecular biological manipulations, and utilized for purification of His-TAT-HA-CARP-1 1-198Y192>F protein. The plasmid pTAT-enhanced green fluorescent protein (eGFP) that encodes His-TAT-HA-tagged eGFP was used.

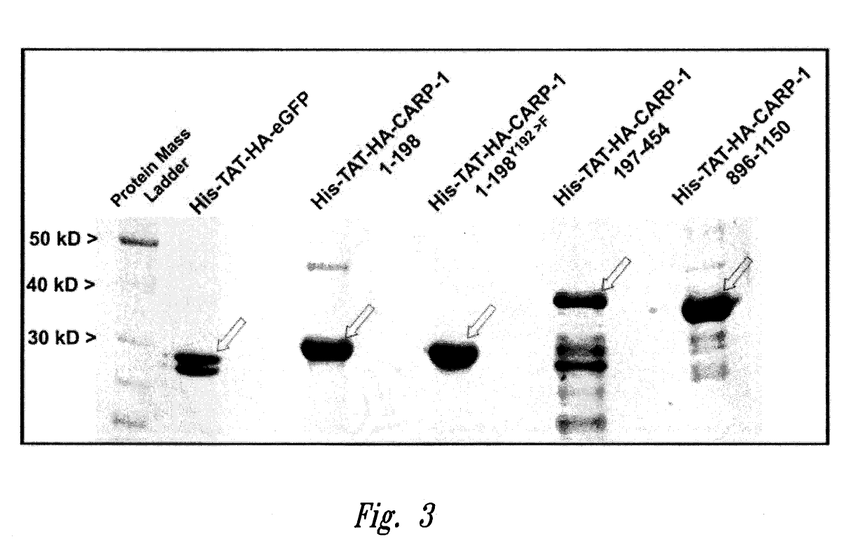

The recombinant plasmids were utilized to transform E. Coli (BL-21) DE3 strain and independent E. Coli isolates expressing respective proteins were obtained. Expression of His-TAT-HA-eGFP, CARP-1 1-198, 1-198Y192>F, 197-454, and 896-1150 proteins was induced by IPTG followed by their affinity purification from bacterial lysates utilizing nickel charged agarose resin (ProBond resin; Invitrogen, Inc.) using manufacturer suggested protocols (FIG. 3).

### Example 2

### Purified TAT-Tagged Proteins Translocate to Perinuclear Region of HBC Cells

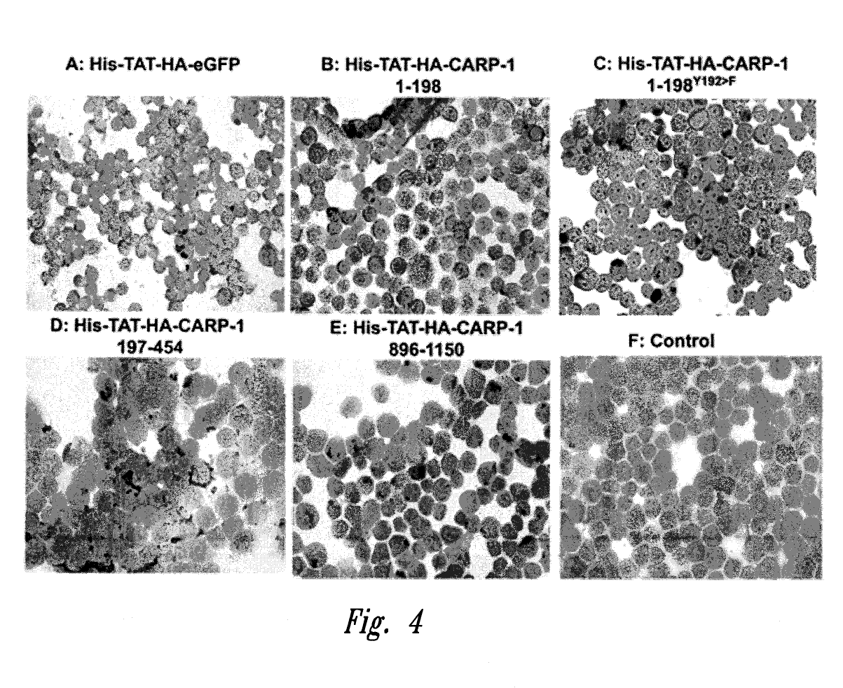

To investigate HBC cell growth inhibitory properties of various TAT-tagged CARP-1 peptides, their transport inside the cells was examined. HBC cells were incubated separately with various affinity-purified peptides, followed by immunocytochemical staining of cells using anti-HA-tag antibodies. As shown in FIG. 4, all the peptides showed significant accumulation in the perinuclear/cytoplasmic region of HBC cells.

### Example 3

### Purified TAT-Tagged CARP-1 Proteins Cause Growth Inhibition in HBC and Lymphoma Cells

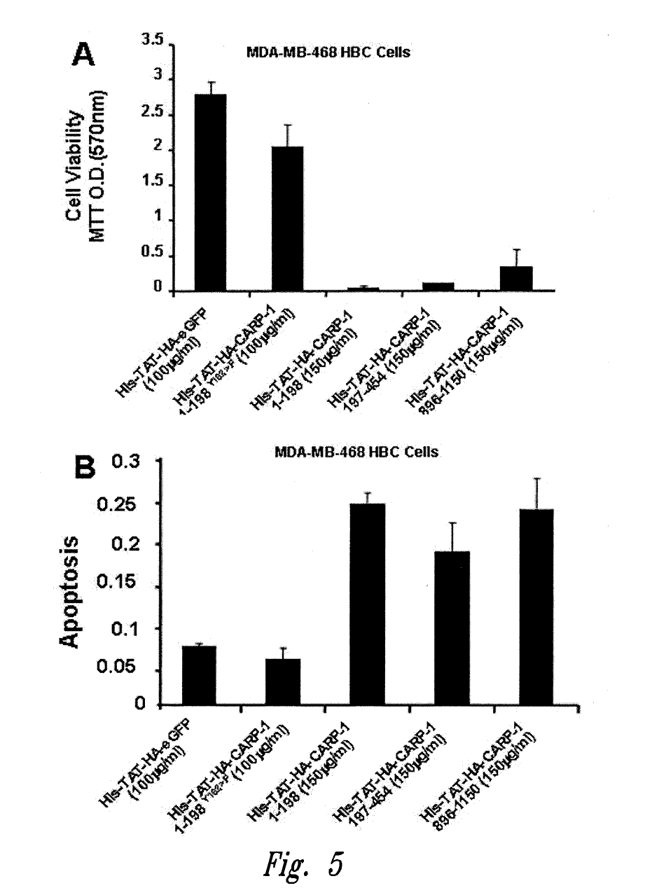

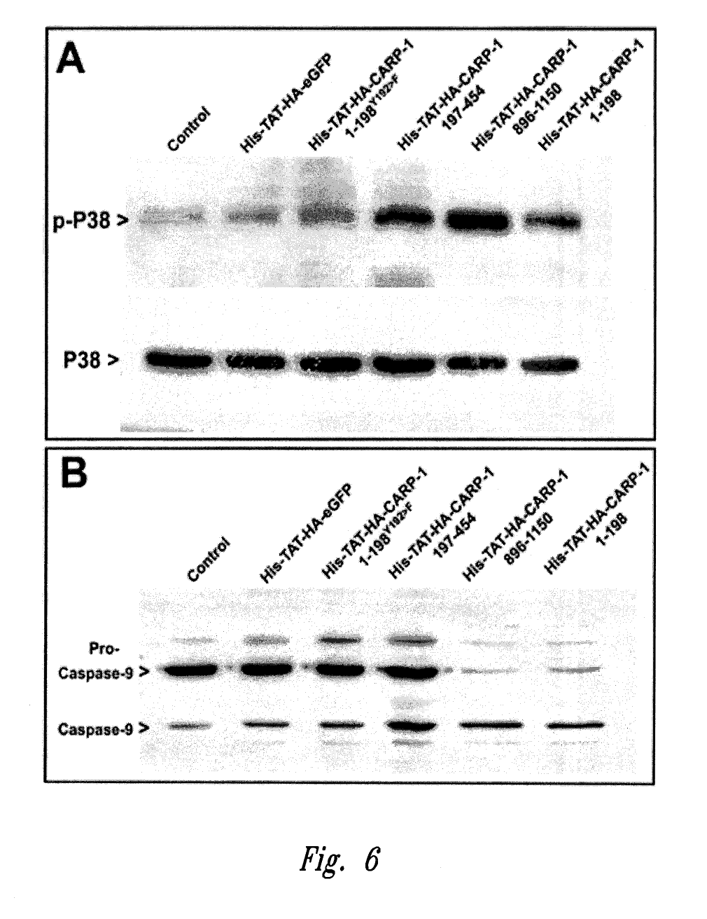

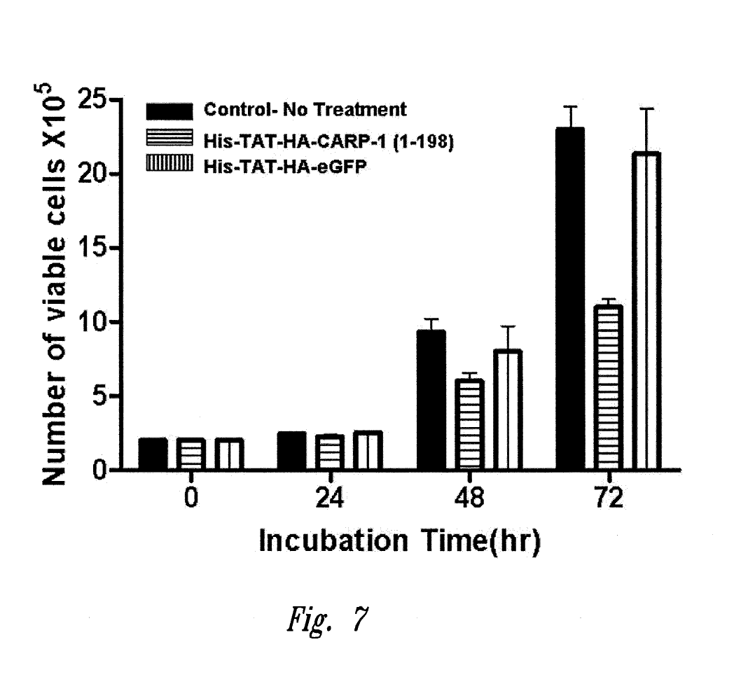

HBC cells were incubated with respective proteins for various time periods followed by determination of cell viability (as a measure of cell growth) and levels of apoptosis. As shown in FIG. 5, incubation with His-TAT-HA-CARP-1 1-198, 197-454 or 896-1150 peptides resulted in loss of viability (panel A) and increased apoptosis (panel B) of HBC cells when compared to their counterparts that were treated with either His-TAT-HA-eGFP or His-TAT-HA-CARP-1 1-198 Y192>F proteins. The apoptosis signaling by these CARP-1 peptides involves activation of pro-apoptotic proteins p38 MAPK and caspase-9 (FIG. 6). Incubation with His-TAT-HA-CARP-1 1-198 peptide also resulted in reduced viability of chemotherapy-resistant lymphoma cells (FIG. 7). The in vitro data described above suggest that the His-TAT-HA-CARP-1 1-198 peptide possesses cell growth inhibitory as well as tumor suppressor properties, and may serve as a potential therapeutic agent for breast and other cancers.

### Example 4

### His-TAT-HA-CARP-1 1-198 Protein Inhibits Growth of HBC Cell-Derived Tumor Xenografts in SCID Mice

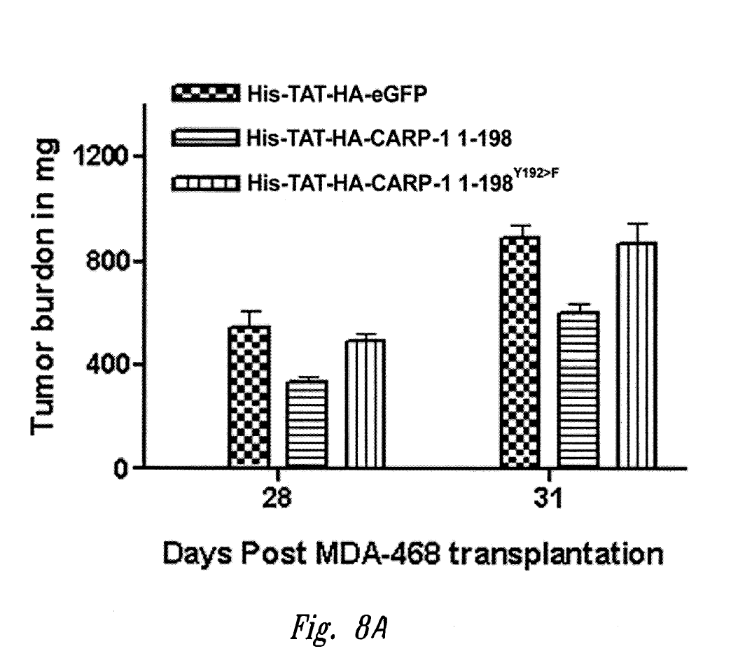

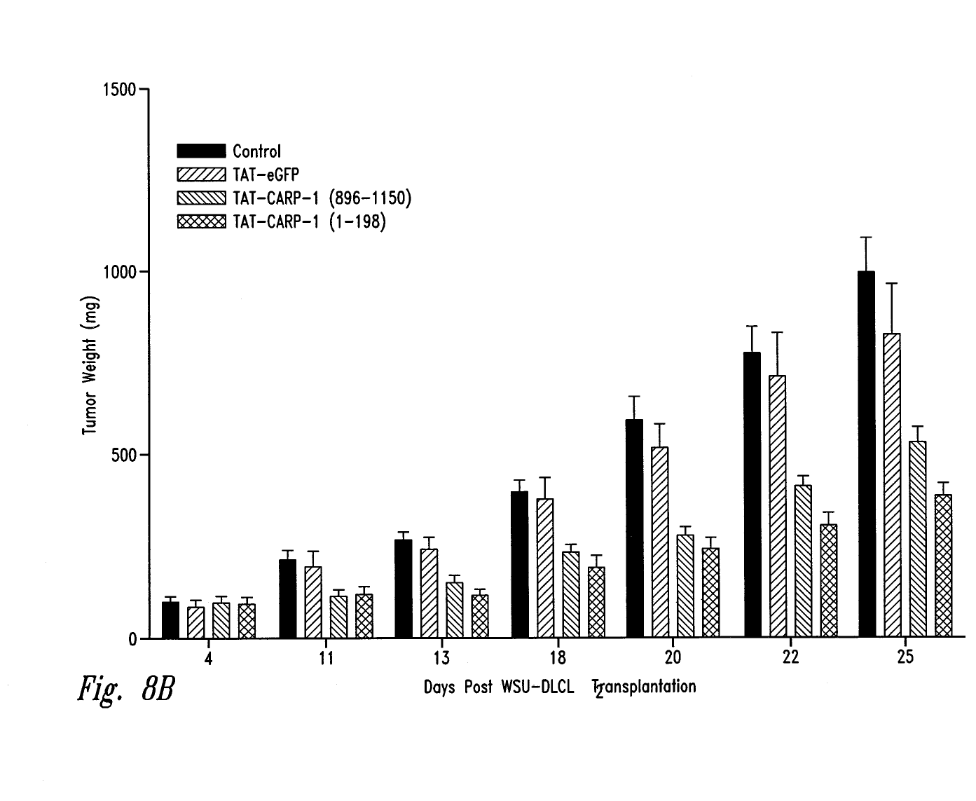

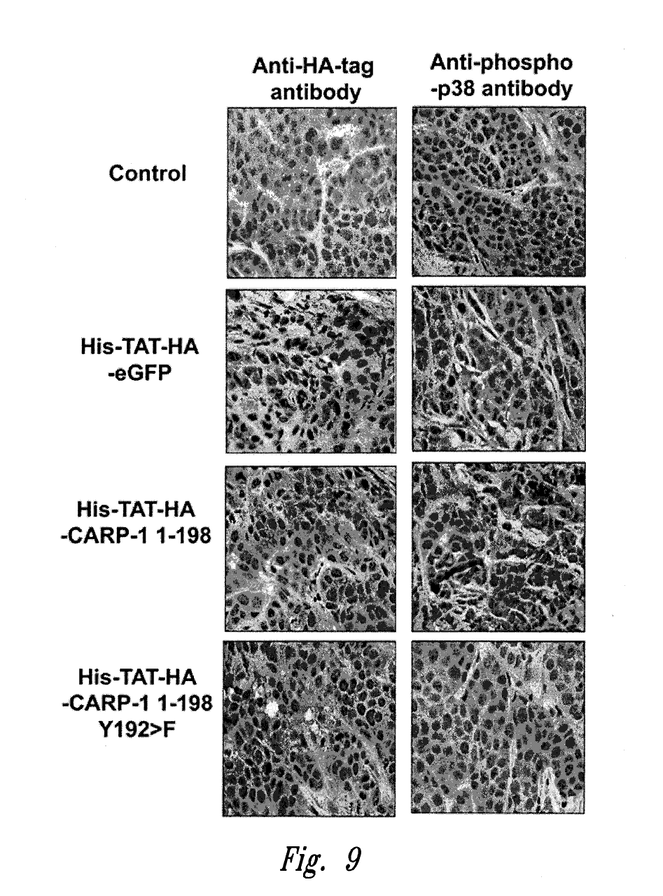

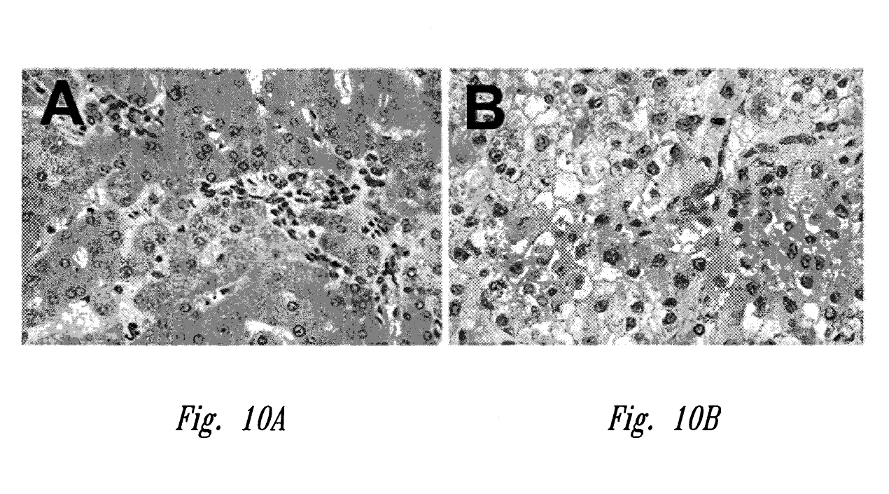

Wild-type MDA-MB-468 HBC cells were first grown as tumors in SCID mice. The tumor fragments were subsequently xenografted bilaterally on the flanks of fresh groups of mice for efficacy studies. After the tumors became visible, four groups of seven mice each were treated either with His-TAT-HA-eGFP, His-TAT-HA-CARP-1 1-198 or His-TAT-HA-CARP-1 1-198 Y192>F proteins. The proteins were injected intratumorally at a dose of 25 μg/tumor/day for five consecutive days, and the tumor growth was monitored for an additional 20 days. The tumor measurements were carried out at multiple time points during the course of treatments and observation periods. The data presented in FIG. 8A are derived from three independent sets of experiments carried out in duplicate, and demonstrate that His-TAT-HA-CARP-1 1-198 caused significant inhibition of tumor growth when compared with the growth of tumors that were treated with either His-TAT-HA-eGFP or His-TAT-HA-CARP-1 1-198Y192>F protein.

### Example 5

### His-TAT-HA-CARP-1 (1-198) and (896-1150) Proteins Inhibited Growth of Drug-Resistant WSU-DLCL2 Lymphoma Cell-Derived Tumor Xenografts in SCID Mice

This example demonstrates that His-TAT-HA-CARP-1 (1-198) as well as (896-1150) proteins inhibited growth of drug-resistant WSU-DLCL2 lymphoma cell-derived tumor xenografts in SCID mice.

Once palpable tumors developed (usually <100 mg in size) by day seven of xenograft implantation, groups of six mice were removed randomly for the efficacy trial using affinity purified TAT-tagged proteins. The proteins were injected intra-tumorally at a dose of 25 mg/tumor/day for five consecutive days, and the tumor growth was monitored for an additional 20 days. The tumor measurements were carried out at multiple time points during the course of treatments and observation periods. Mice were observed for changes in weight and side effects followed by measurement of tumors three times per week. Tumor weights in SCID mice were calculated by the formula: Tumor weight (mg)=(A×B2)/2, where A and B are the tumor length and width (in mm), respectively. The histograms in FIG. 8B show tumor weight (in mg; mean+/−SD).

### Example 6

### Immunohistological Staining of Cancer Samples

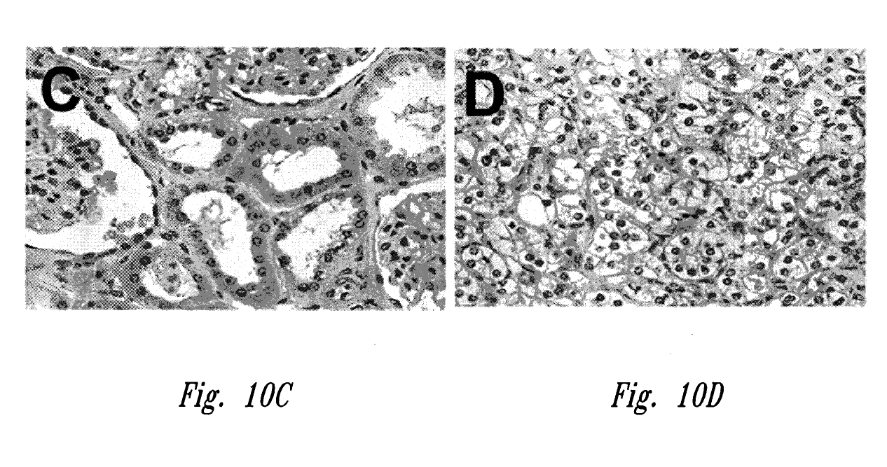

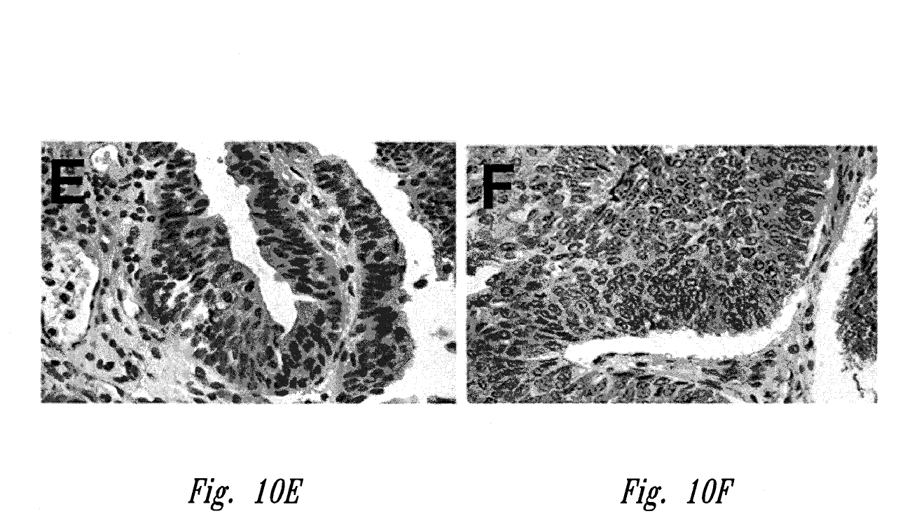

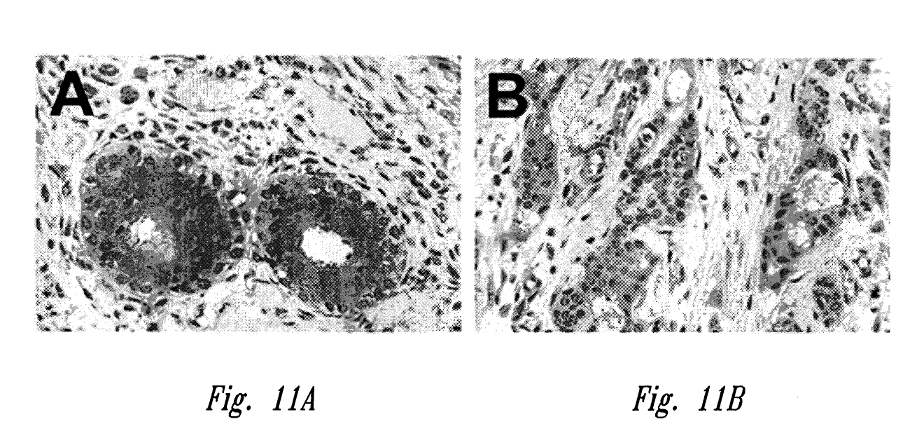

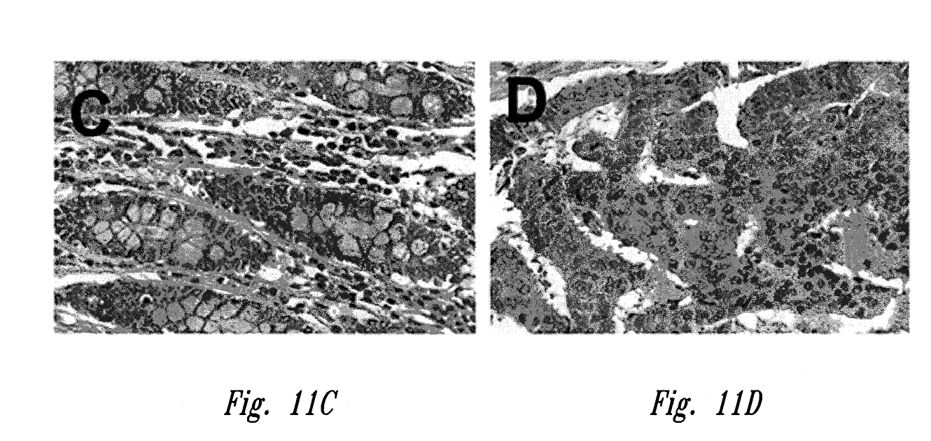

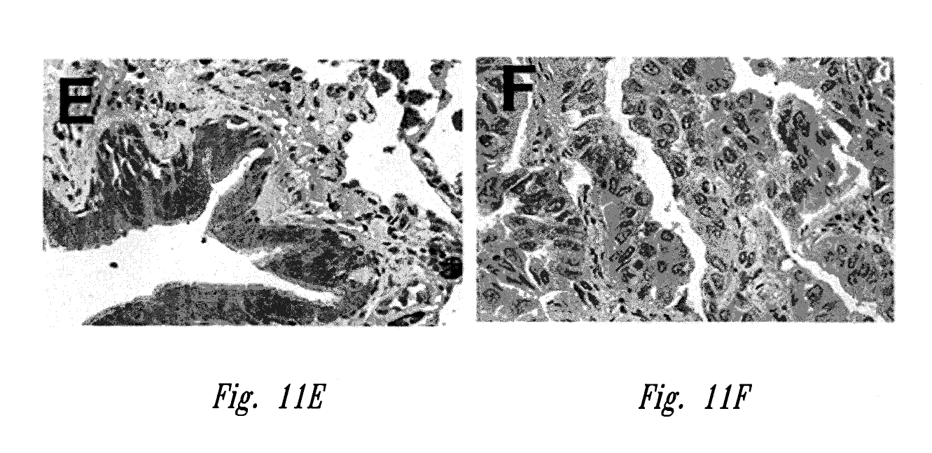

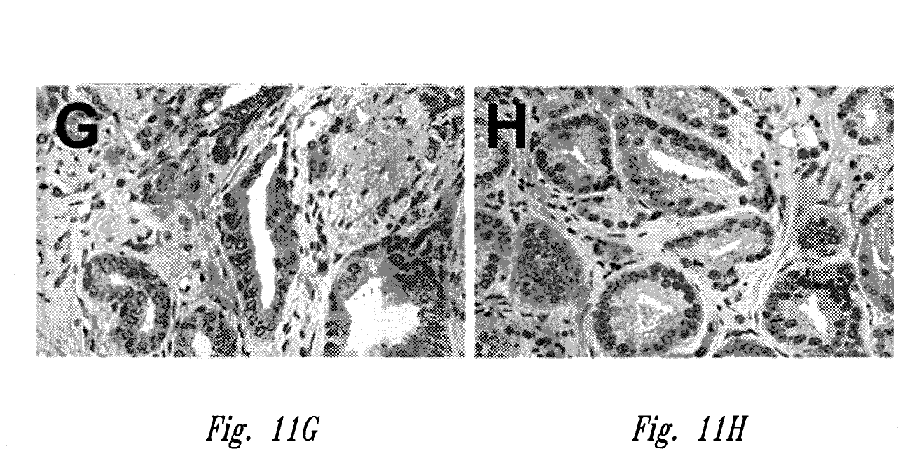

Immunohistological staining of cancer samples is shown in FIG. 10A-F and FIG. 11A-H. Cancer array slides were purchased from Cyberdi, Inc., Gaithersburg, Md. and Petagen Inc., Seoul, South Korea. The tumor specimens were immunostained with anti-CARP-1 (alpha 1) polyclonal antibodies following standard procedures.

FIGS. 10A and B, show liver; 10C and D, kidney; and 10E and F, bladder. Representative higher power (400×) micrographs of poorly differentiated tumors (panels B, D, F) and the adjoining normal tissues (panels A, C, E) are presented.

FIGS. 11A and B show breast; 11C and D, colon; 11E and F, lung; and 11G and H, prostate. Representative higher power (400×) micrographs of poorly differentiated tumors (panels B, D, F, H) and the adjoining normal tissues (panels A, C, E, G) are presented. The darker stained areas in FIGS. 10 and 11 denote CARP-1 presence.

While the description above refers to particular embodiments of the present invention, it will be understood that many modifications may be made without departing from the spirit thereof. The presently disclosed embodiments are therefore to be considered in all respects as illustrative and not restrictive.

1. A method of inhibiting cancer cell growth comprising treating said cancer cell with a composition comprising at least one of His-TAT-HA-CARP-1 1-198 peptide and His-TAT-HA-CARP-1 896-1150 peptide.

2. The method of claim 1, wherein said composition further comprises at least one cancer chemotherapeutic agent.

3-5. (canceled)

6. The method of claim 1, wherein said cancer cell is a breast cancer cell, colon cancer cell, lung cancer cell, prostate cancer cell, liver cancer cell, kidney cancer cell, lymphoma cell, or bladder cancer cell.

7. A pharmaceutical composition comprising a polypeptide consisting of a polypeptide fragment of CARP-1, in admixture with a pharmaceutically acceptable carrier.

8. The pharmaceutical composition of claim 7, wherein said polypeptide consists of contiguous amino acids 1-198 of CARP-1.

9. The pharmaceutical composition of claim 7, wherein said polypeptide is selected from the group consisting of contiguous amino acids 197-454 and 896-1150 of CARP-1.

10. The pharmaceutical composition of claim 7, further comprising at least one cancer chemotherapeutic agent.

11. The pharmaceutical composition of claim 7, wherein said chemotherapeutic agent is adriamycin or Herceptin.

12. (canceled)

13. A method of inhibiting cancer cell growth in a mammalian subject in need thereof, comprising administering a therapeutically effective amount of the composition of claim 7 to the subject.

14. (canceled)

15. The method of claim 13, wherein said mammalian subject is a human afflicted with breast cancer, colon cancer, lung cancer, prostate cancer, liver cancer, kidney cancer, lymphoma, or bladder cancer.

16. (canceled)

17. A method of inducing apoptosis in a cancer cell, said method comprising administering to said cancer cell a polypeptide consisting of contiguous amino acids 1-198 of CARP-1.

18. A method of inducing apoptosis in a cancer cell, said method comprising administering to said cancer cell a polypeptide selected from the group consisting of contiguous amino acids 197-454 and 896-1150 of CARP-1.

19. A method of inducing apoptosis in a cancer cell, said method comprising administering to said cancer cell a vector comprising a polynucleotide encoding a polypeptide consisting of contiguous amino acids 1-198 of CARP-1, wherein the vector is capable of directing expression of said polypeptide in the cell.

20. A method of inducing apoptosis in a cancer cell, said method comprising administering to said cancer cell a vector comprising a polynucleotide encoding a polypeptide selected from the group consisting of contiguous amino acids 197-454 and 896-1150 of CARP-1, wherein the vector is capable of directing expression of said polypeptide in the cell.

21. A method of inhibiting cancer cell growth in a mammalian subject in need thereof, comprising administering a therapeutically effective amount of a vector comprising a polynucleotide encoding a polypeptide consisting of contiguous amino acids 1-198 of CARP-1, wherein the vector is capable of directing expression of said polypeptide in the subject.

22. A method of inhibiting cancer cell growth in a mammalian subject in need thereof, comprising administering a therapeutically effective amount of a vector comprising a polynucleotide encoding a polypeptide selected from the group consisting of contiguous amino acids 197-454 and 896-1150 of CARP-1, wherein the vector is capable of directing expression of said polypeptide in the subject.

23. The method of claim 21, wherein the vector is an AAV vector.

24. The method of claim 21, wherein said mammalian subject is a human afflicted with breast cancer, colon cancer, lung cancer, prostate cancer, liver cancer, kidney cancer, lymphoma, or bladder cancer.

25. The method of claim 22, wherein said mammalian subject is a human afflicted with breast cancer, colon cancer, lung cancer, prostate cancer, liver cancer, kidney cancer, lymphoma, or bladder cancer.

26. (canceled)

27. The method of claim 22, wherein the vector is an AAV vector.
